(19) United States
(12) Patent Application Publication
(10) Pub. No.: US 2007/0293878 A1
(43) Pub. Date: Dec. 20, 2007

(54) MAGNETIC MESH SUPPORT FOR TISSUE WALLS

(76) Inventor: John L. Butsch, Oak Park, IL (US)

Correspondence Address: GODFREY & KAHN S.C., 780 NORTH WATER STREET, MILWAUKEE, WI, 53202, US

(21) Appl. No.: 11/424,707

(22) Filed: Jun. 16, 2006

(51) Int. Cl. A61F 2/02

(52) U.S. Cl. 606/151; 623/23.74

(57) ABSTRACT

A tissue wall support incorporating magnetic elements with a mesh body, and a method of holding a tissue wall support to one side of the tissue wall using a magnet on the opposite side of the tissue wall. The support may be placed adjacent one side of the tissue wall and a magnet may be placed on an opposite side of the tissue wall to draw the support into contact with the tissue wall. Once the support is in contact with the tissue wall, the support can be fixed to that side of the tissue wall.

### BACKGROUND

Wounds, defects and other openings or tears in the abdominal wall of humans and other living creatures require repair and a variety of conventional techniques and devices have been used to close and/or support such openings. Conventional techniques and devices include staples, sutures, and mesh, which are commonly known and widely used in surgical procedures. However, these are not satisfactory for all situations and may result in permanent pain and discomfort. In addition, where defects exist which do not extend entirely through a muscular wall or other corporal feature, sutures or staples may be less than ideal solutions to providing support to the defect to prevent eruption. Improvements to the techniques used to close and support defects or openings in the abdominal wall and other corporal features are desirable.

Laparoscopic surgical techniques are becoming more widely used as an alternative to more traditional surgery involving access through the abdominal wall. These surgical techniques do not result in the same large opening through the abdominal wall but do result in the creation of one or more smaller openings through which the surgical instruments and imaging equipment are inserted. These smaller openings may result in hernias as well. Using laparoscopic equipment to repair hernias has become evermore prevalent in surgical society because it results in less wound infections and less obstructions postoperatively. Repairing hernias laparoscopically has several inherent difficulties. The first of these difficulties is that the placement of the mesh may be cumbersome and some difficult manipulation may be required to get the mesh in place; secondly the securing of the mesh is not perfected and can lead to a permanent sense of discomfort for the patient. Improvements to positioning of the mesh and fixating the mesh to the abdominal wall are desirable.

### SUMMARY

The present invention relates to a tissue wall support incorporating magnetic elements with a mesh body. The support may be placed adjacent one side of the tissue wall and a magnet may be placed on an opposite side of the tissue wall to draw the support into contact with the tissue wall. Once the support is in contact with the tissue wall, the support can be fixed to that side of the tissue wall. The present invention further relates to a method of holding a tissue wall support to one side of the tissue wall using a magnet on the opposite side of the tissue wall.

### BRIEF DESCRIPTION OF THE DRAWINGS

The accompanying drawings, which are incorporated in and constitute a part of the description, illustrate several aspects of the invention and together with the description, serve to explain the principles of the invention. A brief description of the drawings is as follows:

FIG. 1 is a side cross-sectional view of a defect in a tissue wall.

FIG. 2 is a side cross-sectional view of a hollow cannula inserted through the tissue wall with a distal end of the cannula adjacent the defect of FIG. 1.

FIG. 3 is a view, partially in section, of a support according to the present invention ejected from the cannula of FIG. 2 on the distal side of the tissue wall adjacent the defect.

FIG. 4 is a view, partially in section, of the support of FIG. 3 positioned adjacent the distal side of the tissue wall and a magnet adjacent an exterior side of the tissue wall to align and orient the support with respect to the defect.

FIG. 5 is a top view of the support of FIG. 3.

FIG. 6 is a top view of an exterior side of an opening through a tissue wall with a support according to the present invention shown in hidden lines within the tissue wall and mechanical devices used to fix the support in a desired position.

FIG. 7 is a side cross-sectional view of a tissue wall with a support according to the present invention on the distal side of the tissue wall.

FIG. 8 is a top view of a first alternative embodiment of a tissue wall support according to the present invention.

FIG. 9 is a top view of a second alternative embodiment of a tissue wall support according to the present invention.

FIG. 10 is a cross-sectional view of a tissue wall including a defect with a tissue wall support according to the present invention held in place by a pair of subcutaneous magnets.

### DETAILED DESCRIPTION

Reference will now be made in detail to exemplary aspects of the present invention which are illustrated in the accompanying drawings. Wherever possible, the same reference numbers will be used throughout the drawings to refer to the same or like parts.

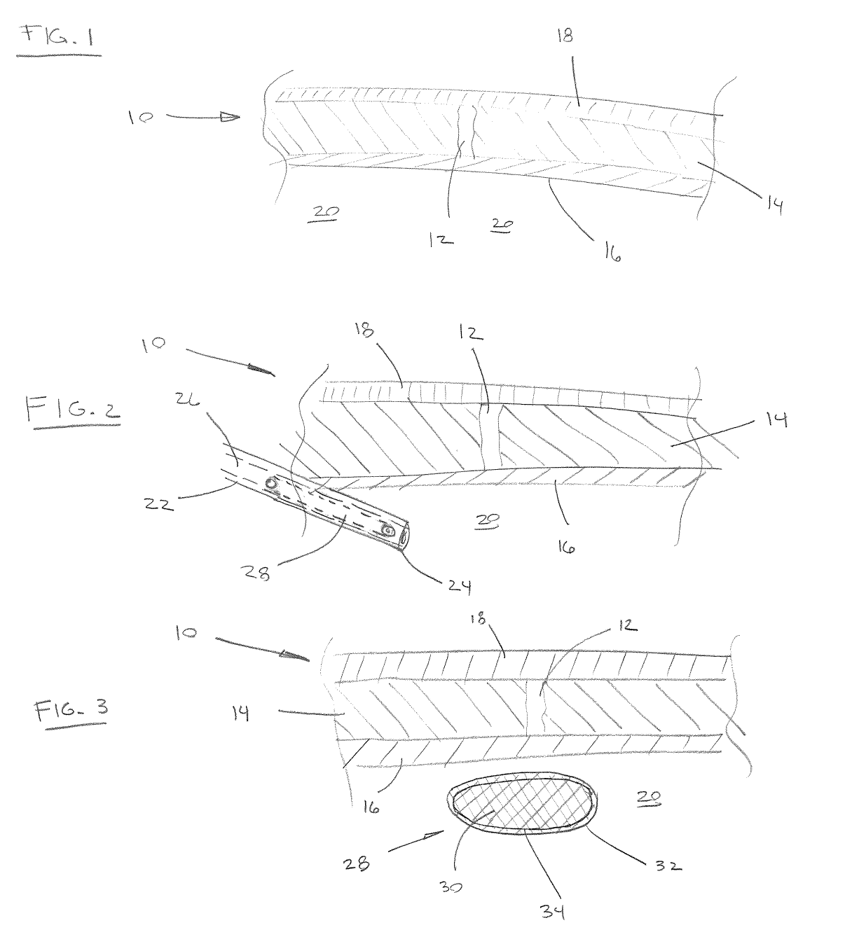

In FIG. 1, a portion of a tissue wall 10 of a living being is shown with a defect 12 visible within at least one layer 14 of the tissue wall. In one example of a tissue wall 10 within a human being, tissue wall 10 may be an abdominal wall and may consist of several layers, such as an inner layer or peritoneum 16, a generally muscular layer 14 and an outer layer 18. While there may be more or fewer layers within tissue wall 10, this description uses a three layer model as representative of a general cross-section of tissue wall 10.

In the human example, defect 12 may be a hernia in the abdominal wall that is desired to be repaired to prevent eruption of any underlying organs (not shown) adjacent inner layer 16. Conventional methodologies for repairing such defects have included both direct and indirect incisions and, in some cases, placement of mesh supports adjacent the defect to provide support to the defect during the healing process. Regardless of the manner in which such a mesh has been placed adjacent inner layer 16, it has not conventionally and conveniently been available to maneuver the support into a desired orientation with respect to the defect and then hold the support in position to permit the support to be fixed to the inner layer.

One approach to placement of such a support in a desired position has been to use laparoscopic techniques in which tools are inserted within a cavity 20 adjacent inner layer 16 of tissue wall 10, as shown in FIG. 2. In FIG. 2, a hollow cannula 22 has been inserted through tissue wall 10 at a site removed from defect 12 and a distal end 24 of the cannula has been positioned adjacent defect 12 and along inner layer 16. Cannula 22 defines a generally cylindrical inner lumen 26 within which may be placed a support 28 in a generally cylindrical form.

Once distal end 24 of cannula 22 has been positioned as desired with regard to defect 12, support 28 may be ejected from lumen 26 and cannula 22 withdrawn from the immediate vicinity of the defect, as shown in FIG. 3. Alternatively, distal end 24 may remain in the immediate vicinity of defect 12 if that might assist in the stabilization or positioning of support 28. In FIG. 3, support 28 has self-expanded to a generally flat form within cavity 20. While it is preferable that support 28 be a self-expanding support that may be collapsed to fit within lumen 26 of cannula 22, support 28 may also be configured to require some assistance from the surgeon to expand within cavity 20. It is also anticipated that support 28 may be introduced into cavity 20 by some other approach than use of cannula 22. For example, some conventional approaches to repairing defects in tissue walls, which approaches may or may not include laparoscopic techniques, utilize the insertion of a support directly through an incision without the use of a cannula or other surgical device.

As shown in FIGS. 3 and 5, support 28 may include a mesh body 30 defining an outer perimeter 32. Positioned about mesh body 30 and adjacent perimeter 32 may be a magnetic element 34. Magnetic element 34 may be made from an inert metallic material, such as a nickel titanium alloy, stainless steel, or some other similar material. However, other metallic and non-metallic materials may also have suitable magnetic and inert qualities that may be suitable or desirable for use as described below. Certain types or formulas of ceramic materials which have magnetic qualities may be used or even metallic materials having weaker magnetic qualities, such as aluminum, may be used. It is not intended to exclude or limit the use of any specific materials that are currently known or may be discovered, provided that support 28 may include the desired magnetic and manipulation abilities to be used in the approaches described below.

In laparoscopic procedures, it is common to have more than one tool in the surgical area to permit tools or other objects to be positioned and also to permit the surgeon to visualize the area and manipulate the tools and other objects. In conventional approaches to laparoscopic repair of defect 12, cannula 22 or another device might be required to position support 28 in its flat form adjacent inner layer 16 to permit the fixing of support to tissue wall 10. In conventional laparoscopic approaches, one or more additional tools may need to be introduced within cavity 20 to manipulate and position support 28 while the surgeon visualizes the area through some form of optical tool.

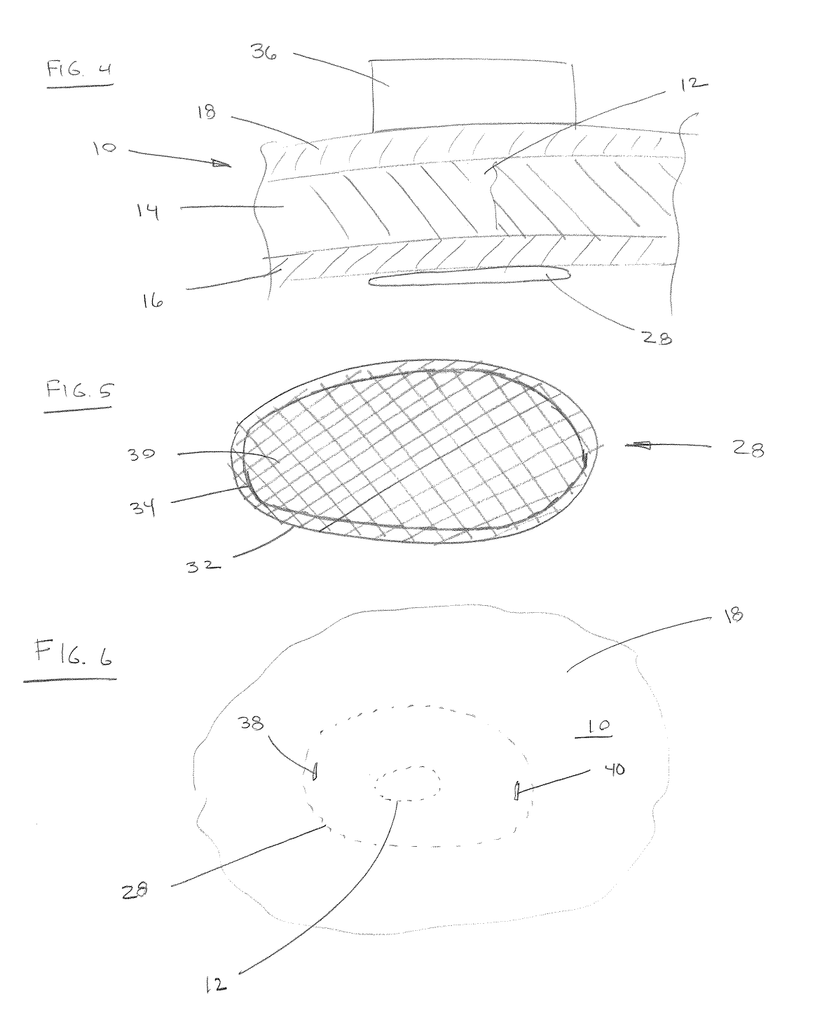

In FIG. 4, instead of requiring a tool within cavity 20 to manipulate and position support 28, a magnet 36 has been positioned adjacent outer layer 18 and has attracted magnetic element 34. It is preferable that magnet 36 be strong enough to attract support 28 through tissue wall 10 and draw support 28 into contact with inner layer 16. Magnet 36 is shown as a block but may be any shape or design which is effective in attracting support 28 into contact with tissue wall 10. Magnet 36 could be a block of magnetic material, which can be oriented to provide greater or lesser amounts of attraction. Alternatively, magnet 36 may be an electromagnet, with the amount of current introduced through the electromagnet determining the amount of pull exerted through tissue wall 10.

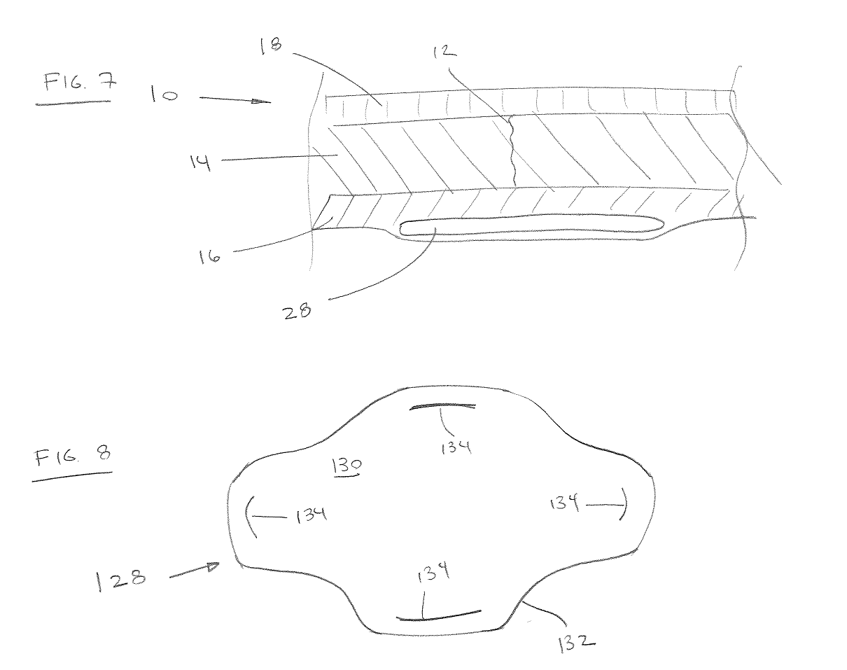

Once support 28 has been brought into contact with tissue wall 10, support 28 is preferably fixed into place along inner layer 16. As shown in FIG. 6, this fixing into position may be accomplished by traditional approaches, such as a surgical staple 38 or a suture 40. One or more of such mechanical fastening approaches may be effected while magnet 36 holds support 28 in position. It is anticipated that other, non-invasive approaches may also be utilized with support 28. In one example, magnet 36 may be left in position adjacent defect 12 and holding support 28 in a desired position in contact with inner layer 16 for a sufficient amount of time for tissue of inner layer 16 to grow through the open material of mesh body 30. Once this tissue through-growth has sufficiently occurred, magnet 36 may be removed from outer layer 18 of tissue wall 10, as shown in FIG. 7. This tissue through-growth may then serve to mechanically fix support 28 to tissue wall 10 without the need for the introduction of additional foreign materials within cavity 20 or extending through all or a portion of tissue wall 10.

As shown in FIG. 6, it may be desirable to have support 28 formed into a particular geometry based on the nature of tissue wall 10 being repaired, the configuration of defect 12 to be repaired, or for other reasons. As such, it may be desirable to ensure that support 28 is oriented in a particular fashion against inner layer 16 of tissue wall 10. In FIG. 6, support 28 is generally oval in shape and defect 12 is elongated in one aspect. It may be desirable to have a major or elongated axis of support 28 generally aligned with the elongated aspect of defect 12. To accomplish this positioning, all or a portion of magnetic element 34 may be polarized, so that magnet 36 will tend to draw support to inner layer 16 in the desired orientation with respect to defect 12.

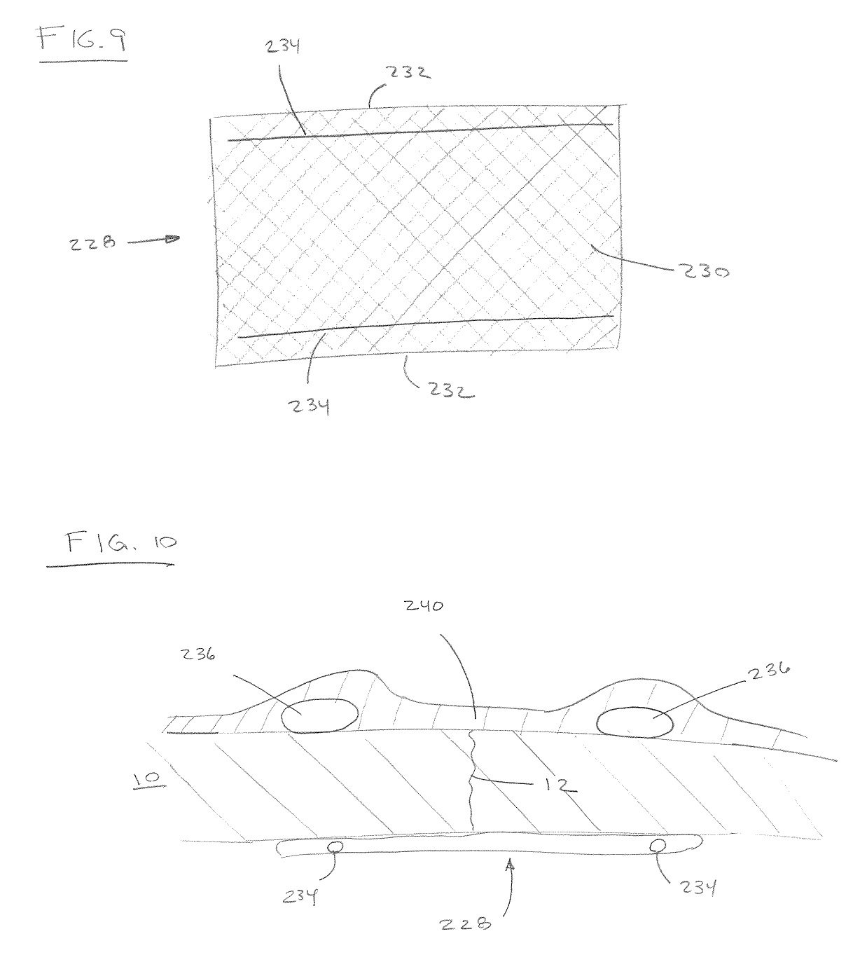

FIG. 8 illustrates an alternative embodiment of tissue support 128. Support 128 includes one or more discrete magnetic elements 134 positioned about mesh body 130. Magnetic elements 134 may be polarized to aid in positioning of support 128, as described above. Support 128 also defines a less regular geometric shape with a scalloped perimeter 132, as the shape of perimeter 132 may be selected to fit a particular application or a particular size or configuration of defect.

FIG. 9 illustrates a second alternative embodiment support 228 which is generally rectangular in shape. Support 228 includes woven material forming a mesh body 230, and intertwined within mesh body 230 are linear magnetic elements 234 running generally parallel to and adjacent sides 230. Magnetic elements 234 on opposite sides of mesh body 230 may be polarized and external magnet 36 may be polarized as well. The polarity of magnetic elements 234 and magnet 36 may cooperate to urge support 228 into an orientation and position chosen by a surgeon or other person placing the mesh. It is anticipated that the polarity of the magnet or the magnetic elements may be permanently configured. Alternatively, the polarity may be temporarily induced by the passing of electrical currents through or about the magnet or the magnetic elements.

Referring now to FIG. 10, in situations where it may be undesirable or impossible to apply sutures or staples to hold support 28, 128 or 228 into position, or where the number of staples or sutures is desired to be reduced, one or magnets 36 may be inserted subcutaneously in a patient. As illustrated, support 228 is positioned adjacent one side of tissue wall 10 and magnets 136 are positioned on the opposite side of tissue wall 10. Magnets 136 are positioned between tissue wall 10 and a dermal layer 240. Thus, instead of having an externally mounted magnet that needs to remain in position for the time required for the support to be incorporated into the tissue wall, sub-dermally mounted magnet 136 may be used, or some combination of external and subcutaneous magnets may be used. After the period of time necessary for adequate incorporation of the support into the tissue wall, the magnet can be removed through an incision and the skin allowed to heal with out any penetration of the inner portions of the tissue wall required.

It is generally known that tissue wall supports such as supports 28, 128 and 228 may need to withstand forces of up to one hundred and fifty Newtons, for example, to properly or adequately protect a hernia in an abdominal wall. This is a typical force that might be exerted by a sudden contractive event affecting the muscles of the abdominal wall, such as a sneeze or a cough. It may be desirable that magnet 36 and magnetic elements 34, 134 and 234 cooperate to provide that level of force holding support 28, 128 or 228 in place against inner layer 16 until the support is sufficiently incorporated into inner layer 16 to withstand this force without external assistance. Alternatively, some combination of mechanical fastening and magnetic attraction may be used to ensure that a support is held sufficiently strongly to the tissue wall in question. The length of time that such fastening and/or attraction is required and the required forces that the support needs to withstand may vary with the nature of the tissue wall being supported and the position of the defect within that tissue wall. It is anticipated that the attractive force between the magnets and the magnetic elements may be varied as required for particular applications and supplemented as needed with more traditional or conventional mechanical fastening techniques.

The size of the openings through mesh body 30, 130 or 230 may also be chosen to adapt to the particular tissue wall to be supported. While the example of repairing an abdominal wall defect has been used above, it is not intended that tissue supports according to the present disclosure are to be limited to such applications. It is anticipated that other muscular tissue walls may be supported using supports 28, 128, 228 or similar embodiments. Defects in other, non-muscular tissue walls could also be repaired using supports such as those disclosed herein. Body 30, 130 or 230 or other embodiments consistent with that described herein, may be made of a variety of materials, such as, but not limited to, polypropylene, expanded polytetraflouroethylene (ePTFE), high density polyethylene (HDPE), or from human or porcine collagen material.

The embodiments of the inventions disclosed herein have been discussed for the purpose of familiarizing the reader with novel aspects of the present invention. Although preferred embodiments have been shown and described, many changes, modifications, and substitutions may be made by one having skill in the art without unnecessarily departing from the spirit and scope of the present invention. Having described preferred aspects and embodiments of the present invention, modifications and equivalents of the disclosed concepts may readily occur to one skilled in the art. However, it is intended that such modifications and equivalents be included within the scope of the claims which are appended hereto.

What is claimed is:

1. A tissue wall support system comprising:
a support comprising:
a generally flat open mesh body formed from a plurality of fibers, the body defining an outer perimeter;
at least one magnetic material included in the mesh of the body adjacent the perimeter;
the mesh body configured for rolling into a cylindrical form and expanding from the cylindrical form to a generally flat form;
a hollow cannula sized to receive the cylindrical form of the support within an interior, the cannula configured to be inserted through the tissue wall from a first side to a second distal side and eject the cylindrical form of the support on the distal side of the tissue wall;
a magnet configured to attract the magnetic material of the support through the tissue wall adjacent a desired location so that the support can be fixed to the distal side of the tissue wall.

2. The tissue wall support system of claim 1, wherein the support is fixed to the distal side of the tissue wall by sutures.

3. The tissue wall support system of claim 1, wherein the support is fixed to the distal side of the tissue wall by staples.

4. The tissue wall support system of claim 1, wherein the support is fixed to the distal side of the tissue wall by growth of the tissue wall through the open mesh of the support.

5. The tissue wall support system of claim 1, wherein the magnetic material of the support is polarized so that the support will be drawn to a particular orientation by the magnet after the support has been ejected on the distal side of the tissue wall and has expanded to the generally flat form.

6. The tissue wall support of claim 5, wherein the polarity of the magnetic material of the support is induced by an electrical current.

7. The tissue wall support of claim 1, wherein the magnet is positioned subcutaneously adjacent the first side of the tissue wall to attract the support to the distal side of the tissue wall.

8. The tissue wall support system of claim 1, wherein the open mesh body of the support is made of fibers including at least one of polypropylene, ePTFE, HDPE, collagen-human material or collagen-porcine material.

9. The tissue wall support system of claim 1, wherein the magnetic elements are formed from at least one of stainless steel, magnetic ceramic compounds, nickel titanium alloy, and aluminum.

10. The tissue wall support system of claim 1, wherein the support is sized to fit about and provide support to a defect in the tissue wall.

11. The tissue wall support system of claim 10, wherein the tissue wall is a muscular wall and the defect is a hernia within the muscular wall.

12. A method of supporting a portion of a tissue wall, the method comprising:
providing a hollow cannula with a tissue wall support in a cylindrical form within an interior of the cannula, the support expandable from the cylindrical form to a generally flat form when ejected from the interior of the cannula, the support having an open mesh design and including a magnetic material within the mesh adjacent a perimeter of the generally flat form;
inserting a distal end of the cannula through the tissue wall from a first side to a second distal side;
ejecting the support from the distal end of the cannula adjacent the portion of the tissue wall to be supported, the support expanding to the generally flat form when ejected from the cannula;
positioning a magnet on the first side of the tissue wall to attract the support into contact with the tissue wall on the distal side of the tissue wall adjacent the portion of the tissue wall to be supported;
holding the support in contact with the tissue wall while the support is fixed to the tissue wall.

13. The method of claim 12, wherein the support is fixed to the tissue wall by suturing.

14. The method of claim 12, wherein the support is fixed to the tissue wall by stapling.

15. The method of claim 12, wherein the support is fixed to the tissue wall by growth of the tissue wall through the open mesh of the support.

16. The method of claim 12, wherein the magnetic material of the support is polarized, and the method further comprises manipulating the magnet adjacent the first side of the tissue wall to orient the support as desired on the distal side of the tissue wall.

17. A tissue wall support comprising:
a generally flat open mesh body formed from a plurality of fibers, the body defining an outer perimeter;
at least one magnetic material included in the mesh of the body adjacent the perimeter;
the mesh body configured for rolling into a collapsed form and self-expanding from the collapsed form to a generally flat form.

18. The tissue wall support of claim 17, wherein the magnetic material of the support is polarized so that the support will be drawn to a particular orientation when exposed to a magnet in the generally flat form.

19. The tissue wall support of claim 18, wherein the polarity of the magnetic material of the support is induced by electrical field.

20. The tissue wall support of claim 17, wherein the open mesh body of the support is made of fibers including at least one of polypropylene, ePTFE, HDPE, collagen-human material or collagen-porcine material.

21. The tissue wall support of claim 17, wherein the magnetic elements are formed from at least one of stainless steel, magnetic ceramic compounds, nickel titanium alloy, and aluminum.

22. A kit for providing support to a tissue wall having a distal side and having a defect in a certain portion of the tissue wall, the kit comprising, in combination:
a tissue wall support comprising:
a generally flat open mesh body formed from a plurality of fibers, the body defining an outer perimeter;
at least one magnetic material included in the mesh of the body adjacent the perimeter;
the mesh body configured for rolling into a collapsed form and self-expanding from the collapsed form to a generally flat form; and
a magnet for co-acting with the magnetic material of the tissue wall support so as to attract the tissue wall support into contact with the distal side of the tissue wall adjacent the portion of the tissue wall having the defect.
